(19) United States
(12) Patent Application Publication
(10) Pub. No.: US 2022/0072128 A1
(43) Pub. Date: Mar. 10, 2022

(54) ULTRASOUND-INDUCED CONVECTION FOR DRUG DELIVERY AND TO DRIVE GLYMPHATIC OR LYMPHATIC FLOWS

(71) Applicant: The Board of Trustees of the Leland Stanford Junior University, Stanford, CA (US)

(72) Inventor: Raag D. Airan, Stanford, CA (US)

(21) Appl. No.: 17/466,302

(22) Filed: Sep. 3, 2021

Related U.S. Application Data
(60) Provisional application No. 63/074,879, filed on Sep. 4, 2020.

(51) Int. Cl. A61K 41/00; A61N 7/00; A61K 9/00

(52) U.S. Cl. CPC A61K 41/0028; A61N 7/00; A61N 2007/0073; A61N 2007/0004; A61N 2007/0021; A61K 9/0019

(57) ABSTRACT

The utility of intrathecal delivery is limited by the poor brain and spinal cord parenchymal uptake of intrathecally delivered agents. A simple noninvasive transcranial ultrasound protocol is provided that significantly increases the brain parenchymal uptake of intrathecally administered drugs and antibodies. This protocol of transcranial ultrasound can accelerate glymphatic fluid transport from the cisternal space into the parenchymal compartment. The low intensity and noninvasive approach of ultrasound in this protocol underscores the ready path to clinical translation of this technique. This low-intensity transcranial ultrasound protocol can be used to directly bypass the blood-brain barrier for whole-brain delivery of a variety of agents. Additionally, this protocol is useful as a means to probe the causal role of the glymphatic system in the variety of disease and physiologic processes to which it has been correlated.

### INTRODUCTION

Drug delivery to the brain is significantly limited by the blood-brain barrier (BBB), which excludes ˜98% of potential small molecule therapeutics and nearly 100% of large therapeutics. In principle, if an agent is administered into the cerebrospinal fluid (CSF) of the cisterns or ventricles of the central nervous system (CNS), e.g. via intrathecal delivery during a spinal tap, the agent would already be across the BBB and therefore able to access the brain and spinal cord parenchyma. While such intrathecal delivery is used for the treatment or prophylaxis of a variety of CSF-based diseases, including leptomeningeal metastatic cancer and infectious meningitis, drug penetration into the CNS parenchyma is known to be severely limited. A means to overcoming this effective CSF-parenchyma barrier could greatly expand the utility of myriad off-the-shelf therapeutic agents for the treatment of numerous CNS diseases.

Recently, researchers have observed that vascular pulsations may drive active transport of cisternal CSF fluid into the interstitial compartment of the brain parenchyma, a system coined the “glymphatic pathway”. While the glymphatic pathway could be utilized for drug delivery, at baseline its rate of fluid transport is insufficient to drive significant convection of intrathecally administered agents into the brain parenchyma. Further, while the glymphatic system has been linked to a variety of physiological states, like sleep, and diseases like Alzheimer's disease or traumatic brain injury, these studies are fundamentally correlative as there are no described means for independently controlling glymphatic transport.

Methods of enhancing delivery of active agents through the glymphatic system are of great interest; addressed by the present disclosure.

### SUMMARY

Methods are provided to utilize low-intensity noninvasive transcranial ultrasound to upregulate the glymphatic pathway to improve the efficacy of intrathecal drug delivery. By applying ultrasound in the appropriate manner and frequency; convective flows are driven into and through the interstitium of a target organ. It is shown herein that noninvasive transcranial low-intensity ultrasound increases parenchymal penetration of intrathecally administered small and large molecular agents, including, for example, chemotherapeutic drugs, antibodies, imaging agents, etc.

In some embodiments, the subject methods and systems provide improved parenchymal delivery of therapeutic agents that are delivered intrathecally via lumbar or cervical puncture. In some embodiments, the subject methods and systems provide improved spread of therapeutic agents in the parenchyma following ultrasound mediated blood-brain barrier opening.

In some cases, the methods include the application of ultrasound with single or multiple transducers. In some cases, the methods include the application of ultrasound with interstitial or intraluminal devices. In some cases, the methods include the use of ultrasound frequencies between 100 kHz to 2 MHz. In certain aspects, the subject methods and systems are paired with invasive needle or catheter-based drug delivery approaches.

In some embodiments, a method is provided for increasing brain penetration of an intrathecally administered agent, the method comprising intrathecally administering an agent to a subject; and applying transcranial ultrasound to the subject to modulate the glymphatic pathway to promote brain penetration of the intrathecally administered agent. The ultrasound can be applied as a low-intensity transcranial scanning ultrasound treatment. Useful ultrasound may have a frequency ranging from 600 kHz to 700 kHz. Useful ultrasound may have an intensity ranging from 10 mW/cm² to 450 mW/cm². Useful ultrasound may have a mechanical index ranging from 0.2 to 0.3.

In some aspects, provided herein is a method of treating or ameliorating a neurological disease or disorder selected from Alzheimer's Disease, epilepsy, tremors, seizures, CNS cancers and tumors (gliomas, glioblastoma multiforme (GBM), medulloblastoma, astrocytoma, diffuse instrinsic pontine glioma (DIPG)), pain, psychiatric diseases (e.g., PTSD, anxiety disorder, depression, bipolar disease, suicidality), traumatic brain injury, sleep disorders, pseudotumor cerebri, and other disorders that may result from a dysfunction of central nervous system glymphatic flows. In certain embodiments, the modulation of the glymphatic pathway with ultrasound as described herein may treat or ameliorate the neurological disease or disorder.

In some embodiments, the composition or method described herein is used in combination with one or more methods of imaging (e.g. fMRI or PET), measuring electrophysiology (e.g. EEG), and/or behavioral assessment of brain function, following focal drug release.

In some aspects, a neurally-active/neuromodulator drug is used as a therapeutic agent, for example and without limitation propofol, ketamine, nicardipine, verapamil, dexmedetomidine, modafinil, doxorubicin, and cisplatin. In some embodiments, the therapeutic agent is a hydrophobic compound. In some embodiments, the therapeutic agent is a vasodilator. In other embodiments the therapeutic agent for delivery include, without limitation. chemotherapeutic agents, such as temozolomide, trimethoprim/sulfamethoxazole, nitrosoureas, procarbazine, vincristine alone or in combination, intrathecal methotrexate, combination chemotherapy (e.g., mechlorethamine, vincristine [Oncovin], procarbazine, plus prednisone [MOPP]), cisplatin, and carboplatin). Antibodies can be delivered, for example antibodies specific for a tumor antigen, antibodies specific for checkpoint inhibitors, co-stimulatory molecules, etc. and other immunomodulatory antibodies, e.g., for example and without limitation panitumumab; nanoparticle encapsulations of therapeutic antibodies or chemotherapy agents, e.g. Abraxane; gene therapy vectors; and the like.

In some embodiments, the subject methods and systems provide for movement of the spinal/epidural anesthesia level to one level higher following catheter placement at a lower level for regional or spinal anesthesia. In some embodiments, the subject methods and systems provide chronic therapies for driving glymphatic flows (e.g. to prevent or treat Alzheimer's) or for driving lymphatic flows (e.g. to treat lymphedema).

In some aspects, provided herein is a method of treating or ameliorating a neurological disease or disorder selected from Alzheimer's Disease, epilepsy, tremors, seizures, CNS cancers and tumors (gliomas, glioblastoma multiforme (GBM), medulloblastoma, astrocytoma, diffuse instrinsic pontine glioma (DIPG)), pain (including neuropathic pain), and psychiatric diseases (e.g., PTSD, anxiety disorder, depression, bipolar disease, suicidality), traumatic brain injury, sleep disorders, pseudotumor cerebri, and other disorders that may result from a dysfunction of central nervous system glymphatic flows.

In certain embodiments, the methods may be used anywhere in the body noninvasively, with the use of lower frequency ultrasound transducers, e.g., focused transducers. In certain embodiments, ultrasound is used to drive lymphatic or glymphatic flows. In some embodiments, the subject methods and systems provide improved spread of therapeutic agents in an organ (e.g. liver) following trans-arterial chemoembolization. In some embodiments, the subject methods and systems provide improved spread of therapeutic agents in an organ following agent infusion following direct organ access with a needle (i.e. as an adjunct to more usual convection enhanced delivery).

In alternative embodiments, intrathecal drug delivery is enhanced with interstitially placed invasive transducers. In certain embodiments, single or multiple ultrasound transducers are coupled to the skin surrounding a target organ or set of organs, for instance, placed around the scalp and spinal column to target the central nervous system. In some embodiments, the drug or therapeutic agent of interest will be placed in the medium perfusing the organ (in the case of vascular delivery) or directly placed in the extracellular fluid of the organ (e.g. cerebrospinal fluid surrounding the organs). At the appropriate time, ultrasound will be applied. The ultrasound pressure field can be varied to achieve the effect by mechanically moving the transducer(s) or by altering the phase timing in the case of multiple transducers. In each case, the same principles may be used to drive lymphatic flows in the body or glymphatic flows in the brain.

These and other objects, advantages, and features of the disclosure will become apparent to those persons skilled in the art upon reading the details of the compositions and methods as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 provides a schematic of ultrasonic glymphatic induction for enhancing the brain penetration of an intrathecally administered agent. A. Following intrathecal injection of an agent into the cisternal cerebrospinal fluid (CSF), to bypass the blood-brain barrier (BBB), transcranial focused ultrasound (FUS) is applied across the intact skull. B. Scanning an ultrasound focus across the whole brain is hypothesized to increase glymphatic transport of cisternal CSF into the brain. The ultrasound focus trajectory is indicated by the dashed line, with the full-width at half maximum of the ultrasound field in orange, and is overlaid onto a maximum intensity projection (MIP) of a 3D volumetric T1w MRI (left; major cerebral arteries in white) and a representative transverse brain atlas section (right) (38), indicating the relevant anatomical structures. Ultrasound protocol: 650 kHz ultrasound frequency, 0.25 MI (0.2 MPa estimated in situ peak negative pressure), held continuously on while scanning the indicated 8×10 mm rectangular trajectory repeatedly for 10 min; estimated local duty cycle of 7.7% given the transverse full-width at half-maximum (FWHM) of the focus being 2.78 mm (longitudinal FWHM 12 mm). It takes about 24 sec to complete one 36 mm loop, and this loop was completed 25 times, which gives a total sonication time of about 10 min.

FIG. 2 shows transcranial ultrasound noninvasively accelerates glymphatic transport into the brain parenchyma for an intrathecally administered 1 kDa MRI tracer. A. Experimental timeline to measure the spread of intrathecally administered MRI contrast agent into the brain using quantitative T1-mapping MRI before and after ultrasound intervention. B. Representative pseudocolor T1w MRI images of rat brains following intrathecal MRI contrast agent injection with sham (top) or ultrasound (bottom) intervention. C-D. Quantitative T1-mapping MRI was used to quantify the contrast agent concentration in the brain before and after intervention. C. Representative T1-maps showing contrast agent in the brain with (bottom) and without (top) transcranial ultrasound application, at 105 min after contrast agent administration and 70 min from the ultrasound intervention; dark regions indicate higher MRI contrast agent concentration. D. Brain volume containing a resolvable amount of the intrathecally administered contrast agent over time showing that the ˜1 kDa contrast agent is driven into a significantly larger volume of brain with the ultrasound intervention, versus sham. Gray column indicates ultrasound intervention timing. Presented as mean±S.D. for groups of n=10-11 (12-105 min) and n=3-4 (180-240 min). *: p≤0.05, **: p≤, 0.01 by ANOVA and post-hoc t-tests, comparing ultrasound to sham (black asterisk) and comparing ultrasound timepoints with the baseline at 12 min (red asterisk). Only the significantly different comparisons are noted.

FIG. 3 shows transcranial ultrasound noninvasively accelerates glymphatic transport into the brain of small (1 kDa) and large (150 kDa) molecule agents following intrathecal administration. A. Experimental timeline to measure ultrasound-induced changes in the brain penetration of intrathecally administered small and large agents. B. Representative pseudocolor near-infrared images of brain slices following cisternal injection of the small molecule tracer (top; ˜1 kDa, IRDye8000W dye) and the large tracer (bottom; ˜150 kDa, Panitumumab-IRDye800). C. Near-infrared fluorescent imaging-defined dye-enhanced area for the small and large molecular tracers revealed that both agents penetrated into the brain to a significantly greater degree with ultrasound, compared to sham. Presented as mean±S.D. for groups of n=3-4 for each agent. *: p≤, 0.05 by two-tailed t-tests, comparing ultrasound to sham.

FIG. 4 shows ultrasonic glymphatic induction is safe. A. Experimental timeline for safety assessment. B. Representative MRI images showing no signs of damage, including edema or hemorrhage, in the brain parenchyma before (left) and after (right) transcranial ultrasound application (trajectory in orange; 0.25 MI in situ, 7.7% local duty cycle for 10 min). C-E. Ex vivo brain slice analysis. C. Representative bright-field image and D. Representative hematoxylin and eosin-stained (H & E) transverse sections of the brain of the same rat as in B. E. Magnified views of the indicated areas of D demonstrating no evidence of brain parenchymal damage with this ultrasound protocol.

FIG. 5 shows optimization of T1-mapping sequence. Measured T1 values within the hippocampal region of the rat brain are not affected by TR ranging between 3000-6000 ms corresponding to 8-25 min long scan times respectively. A. Representative examples of T1-maps with the TR=3000 ms for 8 min scan time, 4000 ms for 18 min scan time, and 6000 ms for 25 min scan time, each taken at 12 min after intrathecal contrast agent injection. Green circles within the hippocampal region are included for the T1 value comparisons. Constant volumes were used across the different sequences for the T1 value comparisons. C. Averaged T1 values across a constant volume (0.37 cm3) for different T1-mapping sequences show that T1 values are not affected by changing TR across these different sequences.

FIG. 6 shows ultrasonic glymphatic induction is safe. A. Representative pseudocolor T1w images of rat brains after cisternal MRI contrast agent injection. Contrast agent uptake was monitored up to 72 hrs given that the CSF is replaced completely by 72 hrs. These results showed that Gd-chelate cleared from the CSF-ISF spaces by 3 hours, and further confirm there is no long-term effects of these interventions. B. T2*w MRI showed no brain parenchymal damage up to 72 hours after intervention. No effects such as edema or hemorrhage were noted at 24 hours (left) or at 72 hours (right) following ultrasound (0.25 MI in situ, 7.7% local duty cycle for 10 min) application.

FIG. 7 shows enhanced brain interstitial/glymphatic flow with ultrasound.

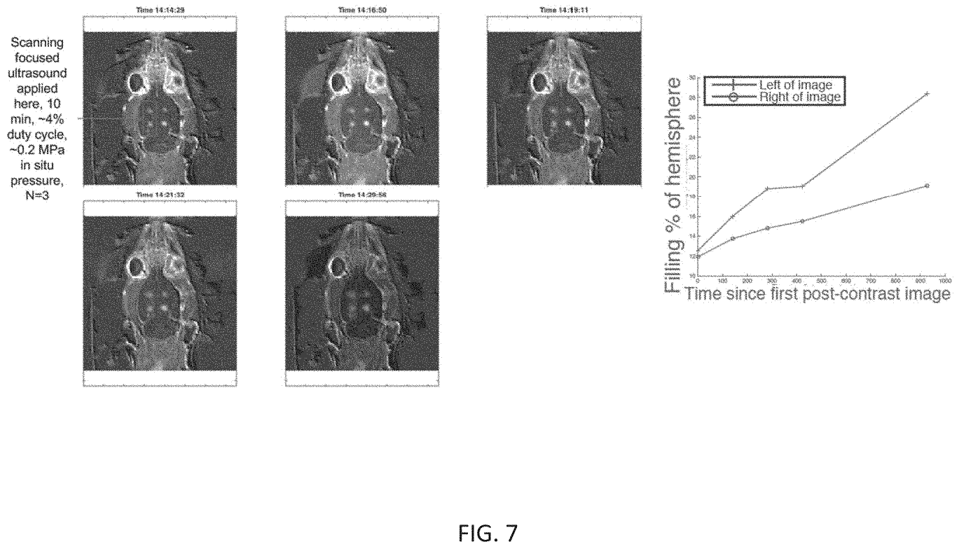

### DETAILED DESCRIPTION

Certain aspects, including embodiments, of the present subject matter may be beneficial alone or in combination, with one or more other aspects or embodiments. Without limiting the following detailed description, certain non-limiting aspects of the disclosure are provided below. As will be apparent to those of skill in the art upon reading this disclosure, each of these aspects may be used or combined with any of the preceding or following aspects. This is intended to provide support for all such combinations of aspects and is not limited to combinations of aspects explicitly provided below.

Ultrasound-mediated drug delivery has gained much attention recently with the availability of clinical focused ultrasound systems that may sonicate any region of the body with millimeter spatial resolution.

Provided herein are methods of increasing delivery of an administered agent to an organ or cells of interest. The methods modulate the interstitial fluid transport systems of the body including, e.g., the glymphatic system or the lymphatic system. The methods can increase delivery of an administered agent with the application of ultrasound, e.g., low intensity or low frequency ultrasound. The ultrasound may be administered to any suitable region of the body, e.g., an organ or and area of the body surrounding the organ. The application of ultrasound may, e.g., accelerate transport of drugs within the interstitium of an organ. In some cases, the agent is administered to a blood vessel or a fluid space near a parenchyma of interest.

In certain embodiments, the methods increase brain penetration of an intrathecally administered agent. By “brain penetration” is meant delivery of an agent to the brain, e.g., to the tissues of the brain. Brain penetration may include the brain and/or spinal cord parenchymal uptake of the agent, e.g., from cisternal cerebrospinal fluid. Brain penetration may be increased with upregulation of the glymphatic pathway. Aspects of the methods may include intrathecally administering an agent to a subject. The methods may further include applying transcranial ultrasound to the subject to modulate the glymphatic pathway to promote brain penetration of the intrathecally administered agent.

As summarized above, the methods may include applying or administering transcranial ultrasound to a subject to modulate the glymphatic pathway to promote brain penetration of the intrathecally administered agent. The term “glymphatic pathway” is used in its conventional sense to refer to a brain-wide network of paravascular channels along which cerebrospinal fluid (CSF) moves into and through the brain parenchyma, facilitating the exchange of CSF and interstitial fluid (ISF) and the clearance of interstitial solutes from the brain. Increasing or promoting the glymphatic clearance system may facilitate clearance of waste products from the brain, such as, e.g., amyloid-β. The glymphatic system was first described by Ilff et al. in 2012 (Ilff et al., Sci Transl Med. 2012; 4:147ra1 1 1). In some cases, the method includes upregulating the glymphatic pathway with the application of ultrasound. In some cases, upregulating the glymphatic pathway includes increasing and/or accelerating glymphatic transport of cisternal cerebrospinal fluid (CSF) into the brain of a subject. In some cases, upregulating the glymphatic transport pathway includes increasing the rate of CSF transport to the brain. The CSF may include an intrathecally administered agent. In some cases, brain penetration of the intrathecally administered agent is increased by a percentage ranging from 50% to 120%, from 50% to 110%, from 50% to 110%, from 60% to 110%, or from 70% to 110% compared to a control. The control may not be subjected to any ultrasound such, e.g., a subject that is not subjected to ultrasound after administration of an agent.

The application of transcranial ultrasound may include the transmission of low-intensity and/or low frequency ultrasound through the skull of a subject. The transcranial ultrasound may be applied in a non-invasive manner. In some cases, the transcranial ultrasound does not produce any tissue damage, e.g., neuronal cell damage, when applied to the brain of a subject. In some cases, the transcranial ultrasound includes transcranial focused ultrasound. Transcranial focused ultrasound may be applied by any convenient means as described in, e.g., U.S. Publication No.'s 2016/0038770 and 2019/0030375, the disclosures of which are incorporated herein by reference in their entireties. The transcranial ultrasound may be applied to any suitable area of the brain and/or spinal cord. In some cases, the transcranial ultrasound is applied or delivered across the whole brain of the subject. In some cases, the transcranial ultrasound is applied or delivered to one or more regions of the brain of the subject.

Ultrasound parameters that may vary include, e.g., ultrasound fundamental frequencies (UFF), intensities (UI), durations (UD), duty cycles (UDC), pulse repetition frequencies (UPRF), mechanical index, etc. The frequency of the applied ultrasound may range from 100 kHz to 700 kHz including, e.g., from 200 kHz to 700 kHz, from 300 kHz to 700 kHz, from 400 kHz to 700 kHz, from 500 kHz to 700 kHz, from 600 kHz to 700 kHz, from 500 kHz to 650 kHz, or from 600 kHz to 650 kHz. In certain embodiments, the applied ultrasound has a frequency that ranges from 100 kHz to 2 MHz including, e.g., 200 kHz to 2 MHz, 300 kHz to 2 MHz, 400 kHz to 2 MHz, 500 kHz to 2 MHz, 600 kHz to 2 MHz, 700 kHz to 2 MHz, 800 kHz to 2 MHz, or 900 kHz to 2 MHz. The ultrasound may be at an intensity in a range of 0.0001 mW/cm² to 100 W/cm². In some cases, the methods include applying low-intensity (<500 mW/cm²) ultrasound. In some cases, the intensity may comprise a range from 10 mW/cm² to 450 mW/cm² including, e.g., from 25 mW/cm² to 450 mW/cm², from 50 mW/cm² to 450 mW/cm², from 100 mW/cm² to 450 mW/cm², from 150 mW/cm² to 450 mW/cm², from 200 mW/cm² to 450 mW/cm², from 250 mW/cm² to 450 mW/cm², from 10 mW/cm² to 400 mW/cm², from 10 mW/cm² to 350 mW/cm², from 10 mW/cm² to 300 mW/cm², from 10 mW/cm² to 250 mW/cm², from 10 mW/cm² to 200 mW/cm², or from 10 mW/cm² to 150 mW/cm². Other intensities that are contemplated include from 1 W/cm2 to 100 W/cm². For example, an acoustic intensity of the methods may comprise 1 W/cm², 2 W/cm², 3 W/cm², 4 W/cm², 5 W/cm², 10 W/cm², 15 W/cm², 20 W/cm², 25 W/cm², 30 W/cm², 40 W/cm², 50 W/cm², 60 W/cm², 70 W/cm², 75 W/cm², 80 W/cm², 90 W/cm², 100 W/cm², or in a range of 10 mW/cm² to 500 mW/cm². The mechanical index of the applied ultrasound may range from 0.1 to 1.9 including, e.g., from 0.1 to 1.5, from 0.1 to 1.0, from 0.1, to 0.5, from 0.1 to 0.3, from 0.2 to 1.9, from 0.2 to 1.5, from 0.2 to 1.0, from 0.2 to 0.5, from 0.2 to 0.4, or from 0.2 to 0.3. Ultrasound may be applied or delivered to a subject for any suitable amount of time ranging from, e.g., 1 minute to 30 minutes, 1 minute to 20 minutes, 1 minute to 15 minutes, 5 minutes to 15 minutes, or 10 minutes to 15 minutes.

As summarized above, the methods may include intrathecally administering an agent to a subject. As used herein, the term “intrathecal administration” or “intrathecal injection” refers to an injection into the spinal canal (intrathecal space surrounding the spinal cord). The intrathecal administering may include administering a pharmaceutical composition directly into the cerebrospinal fluid of a subject, Various techniques may be used including, without limitation, lateral cerebroventricular injection through a burrhole or cisternal or lumbar puncture or the like. In some embodiments, “intrathecal administration” or “intrathecal delivery” according to the present invention refers to IT administration or delivery via the lumbar area or region, i.e., lumbar IT administration or delivery. As used herein, the term “lumbar region” or “lumbar area” refers to the area between the third and fourth lumbar (lower back) vertebrae and, more inclusively, the L2-S1 region of the spine.

In some instances, the agent is a small molecule agent. Naturally occurring or synthetic small molecule compounds of interest include numerous chemical classes, such as organic molecules, e.g., small organic compounds having a molecular weight of more than 50 and less than about 2,500 Daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols.

In some cases, the agent is a protein or fragment thereof or a protein complex. In some cases, the agent is an antibody binding agent or derivative thereof. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. In some cases, the agent is an enzyme or enzyme complex. In some cases, the agent includes a phosphorylating enzyme, e.g., a kinase. In some cases, the agent is a complex including a guide RNA and a CRISPR effector protein, e.g., Cas9, used for targeted cleavage of a nucleic acid.

In some cases, the agent is a nucleic acid. The nucleic acids may include DNA or RNA molecules. In certain embodiments, the nucleic acids modulate, e.g., inhibit or reduce, the activity of a gene or protein, e.g., by reducing or downregulating the expression of the gene. The nucleic acid may be a single stranded or double-stranded and may include modified or unmodified nucleotides or non-nucleotides or various mixtures and combinations thereof. In some cases, the agent includes intracellular gene silencing molecules by way of RNA splicing and molecules that provide an antisense oligonucleotide effect or an RNA interference (RNAi) effect useful for inhibiting gene function. In some cases, gene silencing molecules, such as, e.g., antisense RNA, short temporary RNA (stRNA), double-stranded RNA (dsRNA), small interfering RNA (siRNA), short hairpin RNA (shRNA), microRNA (miRNA), tiny non-coding RNA (tncRNA), snRNA, snoRNA, and other RNAi-like small RNA constructs, may be used to target a protein-coding as well as non-protein-coding genes. In some case, the nucleic acids include aptamers (e.g., spiegelmers). In some cases, the nucleic acids include antisense compounds. In some cases, the nucleic acids include molecules which may be utilized in RNA interference (RNAi) such as double stranded RNA including small interfering RNA (siRNA), locked nucleic acid (LNA) inhibitors, peptide nucleic acid (PNA) inhibitors, etc.

The agent may have any convenient molecular weight. The molecular weight of the agent may range, e.g., from 1 kDa to 250 kDa, from 1 kDa to 200 kDa, or from 1 kDA to 150 kDA. In some cases, the agent is a small molecular agent having a molecular weight ranging, e.g., from 0.1 kDa to 1 kDa or from 0.5 kDa to 1 kDA. In some cases, the agent is a large molecular agent having a molecular weight ranging, e.g., from 10 kDa to 250 kDa, from 10 kDa to 200 kDa, from 10 kDa to 150 kDa, from 10 kDa to 100 kDa, from 50 kDa to 250 kDa, or from 50 kDa to 200 kDa.

The compositions and methods described herein may be useful in basic research and clinical applications where, e.g., the intrathecal delivery of therapeutic agents is desired. An exemplary application for the methods described herein is pharmacotherapy for psychiatric treatment. In certain embodiments, the methods described herein can be used in delivery of epileptogenic treatments.

Another application for the methods described herein is for focal delivery of vasoactive substances to treat alterations of perfusion, e.g. focally delivering calcium channel antagonists like verapamil and/or nicardipine to treat cerebrovascular disorders such as stroke, cerebral vasospasm, or reversible cerebral vasoconstriction syndrome (RCVS).

Another application for the methods described herein is for the focal delivery of therapeutic agents to treat a cardiovascular disease or disorder selected from hypertension, arterial spasm or blockage, cerebral vasospasm, and myocardial or other end organ infarction or ischemia.

Also contemplated is the introduction of the compositions of the present disclosure into the lymphatic system. In certain embodiments, the application of ultrasound may be used to enhance or increase lymphatic flow in the body to aid in therapy of lymphatic disorders such as cancer treatment induced lymphedema.

### Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, some potential and preferred methods and materials are now described. All patents, patent applications and non-patent publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

Where a range of values is provided, it is understood that each intervening value, to the tenth of the unit of the lower limit unless the context clearly dictates otherwise, between the upper and lower limits of that range and any other stated or intervening value in that stated range, is encompassed and specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Furthermore, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used herein, the term “modulating” means increasing, reducing or inhibiting the activity of a biological or physiological pathway. In some cases, “modulate” or “modulating” or “modulation” may be measured using an appropriate in vitro assay, cellular assay or in vivo assay. In some cases, the increase or decrease is 10% or more relative to a reference, e.g., 10% or more, 20% or more, 30% or more, 40% or more, 50% or more, 60% or more, 70% or more, 80% or more, 90% or more, 95% or more, 97% or more, 98% or more, up to 100% relative to a reference. For example, the increase or decrease may be 2 or more times, 3 times or more, 4 times or more, 5 times or more, 6 times or more, 7 times or more, 8 times or more, 9 times or more, 10 times or more, 50 times or more, or 100 times or more relative to a reference.

The well-known “Wada test” (also known as the intracarotid sodium amobarbital procedure (ISAP)) is used to establish the relative contribution of each cerebral hemisphere to language (speech) and memory functions, and is often used before ablative surgery in patients with epilepsy, and sometimes prior to tumor resection. In a majority of subjects, language (speech) is controlled by the left side of the brain. Though generally considered a safe procedure, there are at least minimal risks associated with the angiography procedure that guides the catheter to the internal carotid artery, and thus, researchers are looking into non-invasive ways to determine language and memory laterality-such as fMRI, TMS, magnetoencephalography, and near-infrared spectroscopy.

The blood-brain barrier (BBB) is a system of vascular structures, enzymes, receptors and transporters designed to prevent access of potentially toxic molecules into the CNS, and to enable passage of nutrients, such as glucose, into brain tissues/structures. The continuous capillaries forming the BBB are sealed and have no fenestrations (openings), forming special tight junctions that restrict paracellular transport. Molecules are restricted from passing between the adjacent cells in capillaries of the CNS by these tight junctions, and pinocytosis is also limited across these capillaries; thus, the main mechanism by which molecules/drugs/imaging agents can pass through the capillaries of the CNS into the brain is passive transcellular diffusion. The molecules transported by passive transcellular diffusion are limited to low molecular weight lipophilic molecules, and this permeability of the BBB is proportional to the lipophilicity of the low molecular weight molecules. However, above a certain molecular weight, the permeability of lipophilic molecules across the BBB is substantially reduced.

Compared with the vasculature of many other organs, the normal BBB severely restricts the passage of most drugs from plasma to the extracellular space, with more than an 8-log difference in the entry rate of small, lipid-soluble molecules compared with large proteins. A few macromolecules are able to enter the brain tissue from the blood by a receptor-mediated process; for example, brain cells require a constant supply of iron to maintain their function and the brain may substitute its iron through transcytosis of iron-loaded transferrin (Tf) across the brain microvasculature. Other biologically active proteins, such as insulin and immunoglobulin G, are actively transcytosed through BBB endothelial cells. The presence of receptors involved in the transcytosis of ligands from the blood to the brain offers opportunities for developing new approaches to the delivery of therapeutic compounds across the BBB (Jain, K., (2012) Nanomedicine. 7(8):1225-1233).

Several strategies have been used for manipulating the BBB for drug delivery to the brain, including osmotic and chemical opening of the BBB as well as the use of transport/carriers. However, the drawbacks of such strategies to forcibly open the BBB include causing damage to the barrier and/or allowing uncontrolled passage of drugs or other noxious agents into the brain. Bypassing the BBB by an alternative route of delivery such as transnasal delivery may also be considered. If targeted delivery to brain parenchyma is not the goal, alternative methods for crossing the blood-cerebrospinal fluid barrier may be considered or drugs may be introduced directly in the cerebrospinal fluid pathways by lumbar puncture. Invasive procedures for bypassing the BBB include direct introduction in the brain by surgical procedures. Several potentially effective therapeutic agents for neurological disorders are available but their use is limited because of insufficient delivery across the BBB (Jain, K., (2012) Nanomedicine. 7(8):1225-1233).

In some embodiments, an effective amount of a composition disclosed herein is administered to the subject, and a magnetic resonance image (MRI) of the subject's brain is obtained by imaging the target compound.

In some embodiments, the methods disclosed herein can be combined with methods of imaging (e.g. fMRI), methods of measuring electrophysiology (e.g. EEG), or methods of behavioral assessment of brain function, following, e.g., focal drug release.

A “fluorophore” is a molecule that absorbs light at a characteristic wavelength and then re-emits the light most typically at a characteristic different wavelength. Fluorophores are well known to those of skill in the art and include, but are not limited to rhodamine and rhodamine derivatives, fluorescein and fluorescein derivatives, coumarins and chelators with the lanthanide ion series. A fluorophore is distinguished from a chromophore which absorbs, but does not characteristically re-emit light. “Fluorophore” refers to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′, 5′,7′,1,4-hexachlorofluorescein (HEX), and 2′,7′-dimethoxy-4′,5′-dichloro-6-carboxyfluorescein (JOE); cyanine dyes, e.g. Cy3, CY5, Cy5.5, etc.; dansyl derivatives; 6-carboxytetramethylrhodamine (TAMRA), BODIPY fluorophores, tetrapropano-6-carboxyrhodamine (ROX), ALEXA dyes, Oregon Green, and the like. Combinations of fluorophores also find use, e.g. where transfer or release of a fluorophore leads to a color change.

The compositions disclosed herein may comprise contrast agents to enhance contrast in MRI or fMRI, as well as may be used for analyte detection. The early and widely implemented MRI contrast agents are small-molecule chelates that incorporate paramagnetic ions that alter T1, such as gadolinium (Gd³⁺) or manganese (Mn²⁺ or Mn³⁺). In some embodiments, the contrast agent may comprise gadolinium (Gd). Non-limiting examples of Gd-comprising contrast agents are gadoterate, adodiamide, gadobenate, gadopentetate, gadoteridol, gadoversetamide, gadoxetate, gadobutrol, gadoterate, gadodiamide, gadobenate, gadopentetate, gadoteridol, gadofosveset, gadoversetamide, gadoxetate, and gadobutrol. In some embodiments, the contrast agent comprises 1,4,7,10-tetraazacyclododecane-1,4,7,10-tetraacetic acid (DOTA). In other embodiments, the contrast agent is DOTA-Gd. The contrast agent may be GdNP-DO3A (gadolinium 1-methlyene-(p-NitroPhenol)-1,4,7,10-tetraazacycloDOdecane-4,7,10-triAcetate). In some embodiments, the contrast agent is pH sensitive. For example, 1,4,7,10 tetraazacyclododecane-1,4,7,10-tetraacetic acid (DOTA) may be used for pH sensing. This molecule contains a p-nitrophenol on a twelve-member ring. Under basic conditions, only one water molecule is involved in the coordination, while under acidic conditions, two water molecules will coordinate to Gd. The contrast agent may be an iron oxide, iron platinum, or manganese contrast agent. The contrast agent may be protein contrast agent. The contrast agent should be capable of providing appropriate response to whatever MRI resolution is desired and whatever MRI intensity is used. Additional contrast agents may be found in U.S. Pat. No. 6,321,105, and U.S. Patent Publication US 2015/0202330, each of which is incorporated in their entirety.

Imaging agents can include fluorescent molecules, radioisotopes, nucleotide chromophores, chemiluminescent moieties, magnetic particles, bioluminescent moieties, and combinations thereof. In some embodiments, the composition further comprises a fluorescent dye. The fluorescent dye may be a derivative of rhodamine, erythrosine or fluorescein. The fluorescent dye may be a xanthene derivative dye, an azo dye, a biological stain, or a carotenoid. The xanthene derivative dye may be a fluorene dye, a fluorone dye, or a rhodole dye. The fluorene dye may be a pyronine dye or a rhodamine dye. The pyronine dye may be chosen from pyronine Y and pyronine B. The rhodamine dye may be rhodamine B, rhodamine G and rhodamine WT. The fluorone dye may be fluorescein or fluorescein derivatives. The fluorescein derivative may be phloxine B, rose bengal, or merbromine. The fluorescein derivative may be eosin Y, eosin B, or erythrosine B. The azo dye may be methyl violet, neutral red, para red, amaranth, carmoisine, allura red AC, tartrazine, orange G, ponceau 4R, methyl red, or murexide-ammonium purpurate. Exemplary fluorescent dyes include, but not limited to Methylene Blue, rhodamine B, Rose Bengal, 3-hydroxy-2, 4,5, 7-tetraiodo-6-fluorone, 5, 7-diiodo-3-butoxy-6-fluorone, erythrosin B, Eosin B, ethyl erythrosin, Acridine Orange, 6′-acetyl-4, 5, 6, 7-tetrachloro-2′,4′, 5′, 6′, 7′-tetraiodofluorescein (RBAX), fluorone, calcein, carboxyfluorescein, eosin, erythrosine, fluorescein, fluorescein amidite, fluorescein isothiocyanate, indian yellow, merbromin, basic red 1, basic red 8, solvent red 45, rhodamine 6G, rhodamine B, rhodamine 123, sulforhodamine 101, sulforhodamine B, and Texas Red (sulforhodamine 101 acid chloride).In some embodiments, the compositions and methods disclosed herein may include lipid or protein emulsifiers that improve the stability, drug loading, and drug release efficacy of the system.

The compositions disclosed herein may be administered through any mode of administration. In some aspects, the compositions may be administered intracranially or into the cerebrospinal fluid (CSF). In some aspects, the compositions are suitable for parenteral administration. These compositions may be administered, for example, intraperitoneally, intravenously, or intrathecally. In some aspects, the compositions are injected intravenously. In some embodiments, the compositions are injected into the lymphatic system. In some embodiments, the compositions may be administered enterally or parenterally. Compositions may be administered subcutaneously, intravenously, intramuscularly, intranasally, by inhalation, orally, sublingually, by buccal administration, topically, transdermally, or transmucosally. Compositions may be administered by injection. In some embodiments, compositions are administered by subcutaneous injection, orally, intranasally, by inhalation, into the lymphatic system, or intravenously. In certain embodiments, the compositions disclosed herein are administered by subcutaneous injection.

The terms “individual,” “subject,” “host,” and “patient,” to which administration is contemplated, are used interchangeably herein; these terms typically refer to a mammal, including, but not limited to, murines, simians, humans, mammalian farm animals, mammalian sport animals, and mammalian pets, but can also include commercially relevant birds such as chickens, ducks, geese, quail, and/or turkeys. A mammalian subject may be human or other primate (e.g., cynomolgus monkey, rhesus monkey), or commercially relevant mammals such as cattle, pigs, horses, sheep, goats, cats, and/or dogs. The subject can be a male or female of any age group, e.g., a pediatric subject (e.g., infant, child, adolescent) or adult subject (e.g., young adult, middle-aged adult or senior adult). In some embodiments, the subject may be murine, rodent, lagomorph, feline, canine, porcine, ovine, bovine, equine, or primate. In some embodiments, the subject is a mammal. In some embodiments, the subject is a human. In some embodiments, the subject may be female. In some embodiments, the subject may be male. In some embodiments, the subject may be an infant, child, adolescent or adult.

In some embodiments, disclosed herein is a method of treating or ameliorating one or more symptoms in a model organism that models a neurological disease or disorder selected from Alzheimer's Disease, epilepsy, tremors, seizures, CNS cancers and tumors (gliomas, glioblastoma multiforme (GBM), diffuse instrinsic pontine glioma (DIPG)), pain (including neuropathic pain), and psychiatric diseases (e.g., PTSD, anxiety disorder, depression, bipolar disease, suicidality), wherein the polymeric perfluorocarbon nanoemulsion composition is administered intravenously or into the cerebrospinal fluid (CSF) to the subject/model organism and an uncaging ultrasound pulse is delivered to the subject at an intensity sufficient to yield particle activation (e.g., 1.0 MPa, 50 ms/1 Hz×60 seconds (every second for 60 seconds). In some embodiments, the model organism is a rodent. In some embodiments, the model organism is a rat. In some embodiments, the uncaging ultrasound pulse is delivered to the subject at 1.5 MPa, 50 ms/1 Hz×60 seconds (every second for 60 seconds). In some embodiments, the uncaging ultrasound pulse is delivered to the subject at a pressure between 0.8 and 1.8 MPa, and with a burst length of 10-100 ms. It is to be understood that the method disclosed herein is not limited to the choice of sonication protocol or the specific focused ultrasound transducer, especially because the threshold for activation will be a function of the sonication frequency, the choice of perfluorocarbon, and the particle size.

In some animal model subjects, e.g., rat, a higher frequency of ultrasound is used than may be used in humans. In human subjects, a lower frequency must be used to get through the skull. In some embodiments, disclosed herein is a method of treating or ameliorating one or more symptoms in a subject having a neurological disease or disorder selected from Alzheimer's Disease, epilepsy, tremors, seizures, CNS cancers and tumors (gliomas, glioblastoma multiforme (GBM), diffuse instrinsic pontine glioma (DIPG)), pain (including neuropathic pain), and psychiatric diseases (e.g., PTSD, anxiety disorder, depression, bipolar disease, suicidality), wherein the polymeric perfluorocarbon nanoemulsion composition is administered intravenously or into the cerebrospinal fluid (CSF) of the subject and an uncaging ultrasound pulse delivered to the subject is less than or equal to 1 mega Hz. In some embodiments, subject is a human. In some embodiments, the uncaging ultrasound pulse delivered to the subject is between 220 and 650 kHz. In some embodiments, the uncaging ultrasound pulse delivered to the subject is between 220 and 1000 kHz.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of compounds/therapeutic agents of the present disclosure calculated in an amount sufficient to produce the desired effect in association with a pharmaceutically acceptable diluent, carrier or vehicle.

As used herein, the phrase “pharmaceutically acceptable carrier” refers to a carrier medium that does not interfere with the effectiveness of the biological activity of the active ingredient. Such a carrier medium is essentially chemically inert and nontoxic.

As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, the term “carrier” refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously or into the cerebrospinal fluid (CSF). Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These pharmaceutical compositions can take the form of solutions, suspensions, emulsion, tablets, pills, capsules, powders, sustained-release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic polyamines and lipids, including, but not limited to N-(1(2,3-dioleyloxy)propyl)-N,N,N-trimethylammonium chloride (DOTMA) and diolesylphosphotidylethanolamine (DOPE). Liposomes are suitable carriers for gene therapy uses of the present disclosure. Such pharmaceutical compositions should contain a therapeutically effective amount of the compound, together with a suitable amount of carrier so as to provide the form for proper administration to the subject. The formulation should suit the mode of administration.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “nucleic acid” and “polynucleotide” are used interchangeably herein, and refer to a polymeric form of nucleotides of any length, either deoxyribonucleotides or ribonucleotides, or analogs thereof. Non-limiting examples of nucleic acids and polynucleotides include linear and circular nucleic acids, messenger RNA (mRNA), cDNA, recombinant polynucleotides, vectors, probes, primers, single-, double-, or multi-stranded DNA or RNA, genomic DNA, DNA-RNA hybrids, chemically or biochemically modified, non-natural, or derivatized nucleotide bases, oligonucleotides containing modified or non-natural nucleotide bases (e.g., locked-nucleic acids (LNA) oligonucleotides), and interfering RNAs.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi(dot)nlm(dot)nih(dot)gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “double stranded RNA,” “dsRNA,” “partial-length dsRNA,” “full-length dsRNA,” “synthetic dsRNA,” “in vitro produced dsRNA,” “in vivo produced dsRNA,” “bacterially produced dsRNA,” “isolated dsRNA,” and “purified dsRNA” as used herein refer to nucleic acid molecules capable of being processed to produce a smaller nucleic acid, e.g., a short interfering RNA (siRNA), capable of inhibiting or down regulating gene expression, for example by mediating RNA interference “RNAi” or gene silencing in a sequence-specific manner. Design of a dsRNA or a construct comprising a dsRNA targeted to a gene of interest is routine in the art, see e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; Chuang & Meyerowitz (2000) Proc Natl Acad Sci USA, 97:4985-90; Piccin et al. (2001) Nucleic Acid Res, 29:E55-5; Kondo et al. (2006) Genes Genet Syst, 81:129-34; and Lu et al. (2009) FEBS J, 276:3110-23; the disclosures of which are incorporated herein by reference.

The terms “short interfering RNA”, “siRNA”, and “short interfering nucleic acid” are used interchangeably may refer to short hairpin RNA (shRNA), short interfering oligonucleotide, short interfering nucleic acid, short interfering modified oligonucleotide, chemically-modified siRNA, post-transcriptional gene silencing RNA (ptgsRNA), and other short oligonucleotides useful in mediating an RNAi response. In some instances siRNA may be encoded from DNA comprising a siRNA sequence in vitro or in vivo as described herein. When a particular siRNA is described herein, it will be clear to the ordinary skilled artisan as to where and when a different but equivalently effective interfering nucleic acid may be substituted, e.g., the substation of a short interfering oligonucleotide for a described shRNA and the like.

“Complementary,” as used herein, refers to the capacity for precise pairing between two nucleotides of a polynucleotide (e.g., an antisense polynucleotide) and its corresponding target polynucleotide. For example, if a nucleotide at a particular position of a polynucleotide is capable of hydrogen bonding with a nucleotide at a particular position of a target nucleic acid, then the position of hydrogen bonding between the polynucleotide and the target polynucleotide is considered to be a complementary position. The polynucleotide and the target polynucleotide are complementary to each other when a sufficient number of complementary positions in each molecule are occupied by nucleotides that can hydrogen bond with each other. Thus, “specifically hybridizable” and “complementary” are terms which are used to indicate a sufficient degree of precise pairing or complementarity over a sufficient number of nucleotides such that stable and specific binding occurs between the polynucleotide and a target polynucleotide.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. As such, an antisense polynucleotide which is 18 nucleotides in length having 4 (four) noncomplementary nucleotides which are flanked by two regions of complete complementarity with the target nucleic acid would have 77.8% overall complementarity with the target nucleic acid. Percent complementarity of an oligomeric compound with a region of a target nucleic acid can be determined routinely using BLAST programs (basic local alignment search tools) and PowerBLAST programs known in the art (Altschul et al., J. Mol. Biol, 1990, 215, 403-410; Zhang and Madden, Genome Res, 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math, 1981, 2, 482-489).

The patents, patent applications and publications discussed herein are provided solely for their disclosure prior to the filing date of the present application, and are incorporated by reference herein in their entirety. Nothing disclosed herein is to be construed as an admission that the present disclosure is not entitled to antedate such publication by virtue of prior invention. Further, the dates of publication provided may be different from the actual publication dates which may need to be independently confirmed.

### EXAMPLES

The following examples are set forth to provide those of ordinary skill in the art with a complete disclosure and description of how to make and use the present compositions and methods, and are not intended to limit the scope of what the inventors regard as their invention nor are the examples intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, (e.g., “bp” refers to base pair(s); “kb” refers to kilobase(s); “ml” refers to milliliter(s); “s” or “sec” refers to second(s); “min” refers to minute(s); “h” or “hr” refers to hour(s); “aa” refers to amino acid(s); “nt” refers to nucleotide(s); “i.v.” or “IV” refers to intravascular(ly); and the like.

### Example 1

As the glymphatic system is driven by convective pressures induced by arterial pulsation, and since ultrasound is a high-frequency wave of pressure oscillations in the medium, we hypothesized that ultrasound application could upregulate glymphatic transport and that this could be used to increase the brain parenchymal penetration of intrathecally administered agents. Indeed, several groups have shown that the bioeffect produced by ultrasound may yield increased interstitial convection of agents in a localized brain region using low pressure combined with an exogenous vesicle (microbubble) and relatively high pressure without these vesicles. However, it has remained an open question of whether a brain-wide application of low-intensity ultrasound may indeed increase the cisternal CSF-interstitial transport that is the hallmark of the glymphatic pathway.

Here, we demonstrate that we may indeed use noninvasive transcranial low-intensity ultrasound to increase the parenchymal penetration of intrathecally administered small and large molecular agents.

### Results

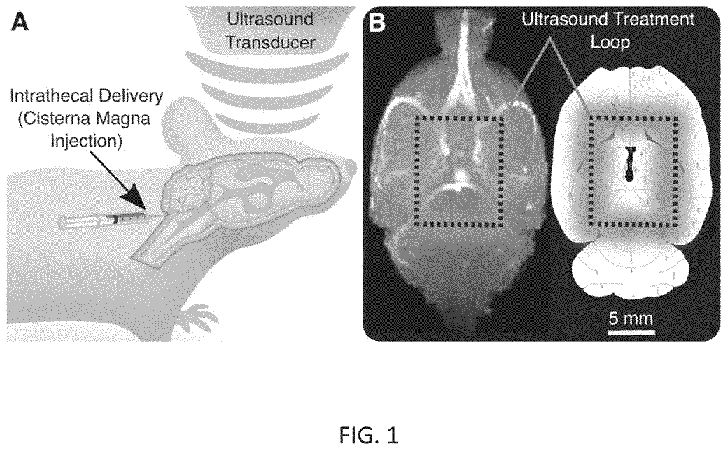

Given the known effects of anesthesia and sleep on glymphatic transport, titration of isoflurane anesthetic dose and environmental heating was used with cardiorespiratory monitoring to ensure physiologic stability during the up to 4 hours of each experiment and intervention, with respiratory rates maintained in the range of 50-60 breaths per minute, heart rates of approximately 300-370 beats per minute, O₂ saturation of approximately 98-100%, and body temperature of 36.5-37.5° C. A scanning ultrasound protocol was chosen to treat the whole rat brain with transcranial focused ultrasound of an intensity below FDA-approved limits for diagnostic ultrasound (0.25 mechanical index, MI, in situ, 7.7% local duty cycle, for a total of 10 min.; FIG. 1). This intensity of ultrasound was chosen as it is less than or similar to the intensities used in routine diagnostic ultrasound imaging of adult and neonatal human patient brains, and is readily achievable with ultrasound systems designed for diagnostic or therapeutic transcranial ultrasound applications in the adult human brain. Notably, the total temperature rise in the sonicated zone due to this level of ultrasound exposures is estimated to be <0.01° C.

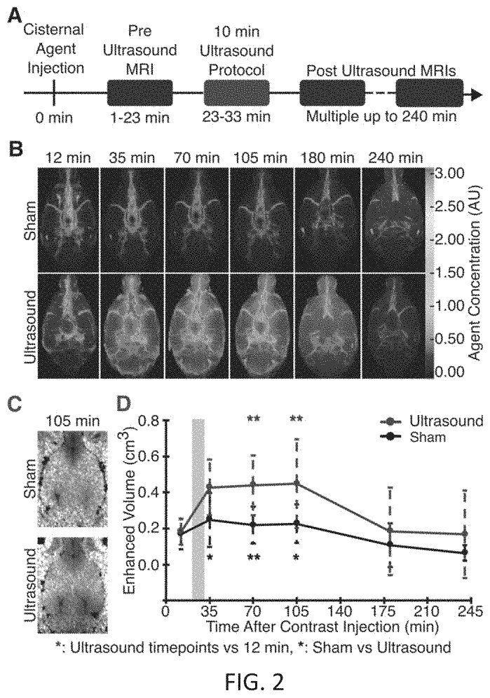

Transcranial ultrasound noninvasively accelerates Qlymphatic transport of a 1 kDa tracer. A gadolinium (Gd)-chelate MRI contrast agent was injected intrathecally into the cisterna magna, to label the CSF, thereby enabling MRI visualization of glymphatic CSF transport from the basal cisterns into the brain parenchyma. 3D T1w MRI images revealed that perivascular influx of CSF into the brain is observed initially 12 min after intrathecal administration of the ˜1 kDa Gd-chelate. Dynamic quantitative T1-mapping MRI imaged the time-dependent CSF influx, parenchymal uptake, and clearance of the Gd-chelate (FIG. 2B). Without further intervention, the ˜1 kDa Gd-chelate enters the brain from the cisterns with a peak brain concentration at approximately 35 min and then it clears from the brain interstitial compartment within 3 h from injection (FIG. 2B). With a low-intensity transcranial scanning ultrasound treatment, a more diffuse pattern of Gd-chelate brain distribution was observed. The peak parenchymal uptake with ultrasound was increased by 72-101% with a relatively delayed peak of 105 min from injection, and with increased residual tracer in the brain at 3 h post-injection (FIG. 2). Notably, the contrast agent entered the brain preferentially near sites of arterial influx into the brain (FIG. 1B, left; FIG. 2), in keeping with known patterns of glymphatic entry into the brain. We noted a statistically significant difference of brain parenchymal tracer uptake in these trends between the ultrasound and sham conditions at 35, 70, and 105 min following tracer administration.

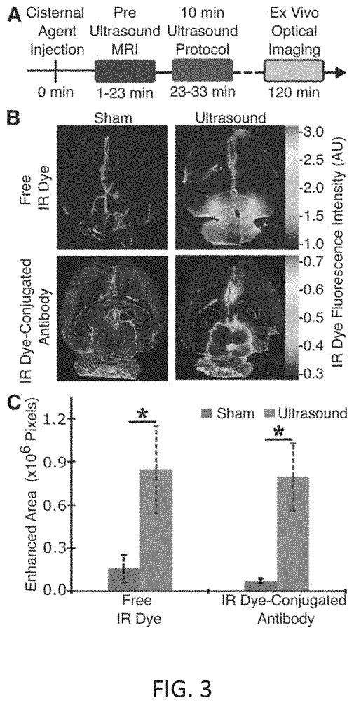

Ultrasound noninvasively increases glymphatic transport of both small and large agents. During Gd-chelate injection, we co-administered an infrared-fluorescent dye (˜1 kDa, IRDye8000W) in free form or the therapeutic antibody panitumumab (˜150 kDa, in active clinical use for EGFR-targeted therapy) conjugated with the same dye to further model the delivery of both small and large therapeutic agents and to be able to validate the MRI findings with an optical imaging modality. Glymphatic upregulation with this ultrasound protocol was confirmed as before using MRI visualization of the Gd-chelate. Animals were sacrificed two hours after agent administration (at peak parenchymal uptake as noted in the initial experiments, FIG. 2D). Histologic infrared fluorescent microscopy verified that both the small and large optical tracers indeed penetrated to a greater degree into the brain with this brief, 10 min noninvasive ultrasound therapy (FIG. 3).

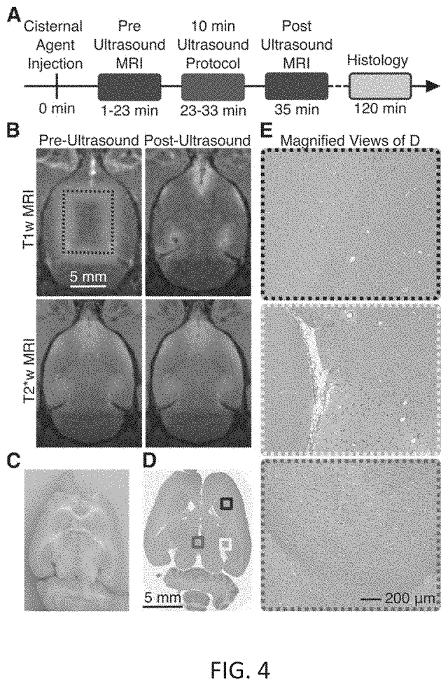

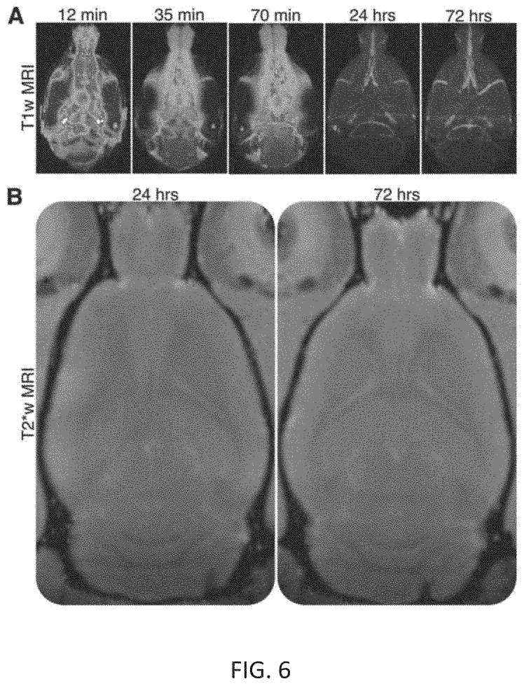

Ultrasonic glymphatic induction is safe. To evaluate the safety of this approach, high-field 7T MRI and histologic evaluation were utilized in both the acute and delayed settings, up to 72 h following ultrasound intervention. No evidence of microhemorrhage or edema was noted using T2*w MRI either within hours of intervention (FIG. 4) or up to 72 h following intervention (FIG. 6). Notably, no prolonged Gd-chelate deposition was seen in the brain up to 72 h, the time by which the CSF is known to be fully replaced (FIG. 6). High-field histological evaluation (FIG. 4C-E) confirmed the lack of brain parenchymal damage with this intervention. Importantly, the total temperature change in the sonicated zone with this ultrasound protocol (0.25 MI in situ, 7.7% local duty cycle for 10 min) is estimated to be <0.01° C. based on the bio-heat transfer equation (Nyborg 1988; Haar and Coussios 2007). Further, this in situ intensity of ultrasound is similar to that used commonly for diagnostic brain imaging in both adult and neonatal human populations, and is well below FDA guidelines for ultrasound application in human tissue. Therefore, this level of safety with this approach is expected.

We have demonstrated that low-intensity noninvasive transcranial ultrasound upregulates the glymphatic pathway to improve the efficacy of intrathecal drug delivery. With MRI, we observed that ultrasound safely accelerates the transport of a ˜1 kDa MRI tracer from the CSF into the interstitial space before it clears from the brain (FIG. 1, 2, 5, Table 1) with a clearance timeline (3 h) consistent with the known pharmacokinetics of intrathecally administered agents. Using optical tracers, we validated the MRI findings using a ˜1 kDa optical tracer that has a similar molecular weight as the MRI tracer (FIG. 3) and which models the distribution of small molecule drugs that are commonly intrathecally administered, like methotrexate. Further, we used the same optical probe conjugated to a ˜150 kDa therapeutic antibody panitumumab and saw similar increases of brain parenchymal uptake of this larger therapeutic agent. Importantly, we saw no evidence of brain parenchymal damage with this approach (FIG. 4, 6).

Overall, our results suggest that low-intensity noninvasive transcranial ultrasound may be used to increase the whole-brain delivery of a variety of small or large therapeutic agents, following the same intrathecal administration that is used routinely in clinics worldwide to administer therapeutic agents into the CSF. Further, this method provides a means to directly upregulate glymphatic transport, which could be used for causative evaluation of the role of the glymphatic system in the variety of physiological and disease processes to which the glymphatic system has been correlated. Given the low intensity of ultrasound necessary for these results, at levels readily achievable with currently-utilized clinical transcranial ultrasound systems, and the lack of need for non-therapeutic exogenous agents for this effect, there is a ready path for clinical translation of a therapy based on these results.

TABLE 1
MRI protocol used for the experiments. MRI protocol used for each
experiment on a Bruker 7T MRI with an IGT single channel transmit/receive coil.

| | | | | | | | Scan | Image |
|---|---|---|---|---|---|---|---|---|
| | | | | | FOV | ST | Time | Resolution |
| Name | Type | TE (ms) | TR (ms) | FA | (mm) | (mm) | (min) | (mm) |
| Localizer | 3 Plane | 2.50 | 26.07 | 30 | 80 × 80 | 1 | 0.06 | 0.313 × 0.313 |
| FLASH- | Coronal | 3.53 | 50.00 | 20 | 60 × 40 × 15 | 0.3 | 6.37 | 0.313 × 0.267 × 0.313 |
| T1-3D | | | | | | | | |
| T2*map- | Coronal | 3.50, 8.5, 13.5, 18.5, | 796.76 | 50 | 60 × 40 | 1 | 4.44 | 0.234 × 0.156 |
| MGE | | 23.5, 28.5, 33.5, | | | | | | |
| | | 38.5, 43.5, 48.5 | | | | | | |
| T1-map | Coronal | 7.00 | 300, 600, 1000, | | 35 × 31 | 1 | 8 | 0.273 × 0.242 |
| RARE | | | 1500, 2000, 3000 | | | | | |
| | | | 300, 600, 1000, | | | | 18 | |
| | | | 1500, 2000, 4000 | | | | | |
| | | | 300, 600, 1000, | | | | 25 | |
| | | | 1580, 2000, 6000 | | | | | |

TE: Echo Time;
TR: Repetition Time;
FA: Flip Angle;
FOV: Field of View;
ST: Slice Thickness;
RARE: Rapid Acquisition with Relaxation Enhancement;
FLASH: Fast Low Angle Shot;
MGE: Multiple Gradient Echoes

### Methods and Materials

Animals. The Institutional Animal Care and Use Committees of Stanford University approved all animal experiments. Tests were performed in 42 male Long-Evans rats with bodyweight 300-350 gm (Charles River Laboratories, Wilmington, Mass., USA). Animals were randomly assigned to one of two groups: (1) no treatment (Sham), and (2) treatment (Ultrasound). Ultrasound (0.25 MI in situ, 7.7% duty cycle for 10 min) or sham was applied transcranially throughout the brain (FIG. 1A). Before each procedure, the fur on the neck was shaved and a cisterna magna injection of a gadolinium (Gd) chelate (Multihance, Bracco Diagnostics, NJ, USA) was performed while the animal was anesthetized under isoflurane. The body temperature, cardiac and respiratory rates, and O₂ saturation were monitored throughout the experiment and the isoflurane level was titrated to keep these parameters constant; environmental heating was used to help maintain body temperature. Localizer, FLASH-T1-3D, T1-mapping, and T2*-weighted MR images were taken to visualize glymphatic transport across the brain, to quantify Gd-chelate kinetic parameters, and to evaluate for parenchymal damage. In separate cohorts, either of two different sized optical tracers, a small molecule (IR800CW Carboxylate, LICOR, Lincoln, Nebr., USA; ˜1 kDa) or a large molecule (Panitumumab-IRDye800: ˜150 kDa, 5 nM, produced under GMP at the Leidos Biomedical Research Center, Frederick, Md., USA) (39) were co-delivered with the Gd-chelate to model the delivery of similar-sized therapeutic agents.

Intrathecal Cisterna Magna Injection For anesthesia, the animals were induced with 5% isoflurane in oxygen using an induction chamber and then switched to a maintenance dose of 2%. The animal was positioned in a stereotaxic frame (Stoelting, Wood Dale, Ill., USA), immobilized with ear bars, and then the head flexed to 45 degrees. A 27-gauge catheter (Butterfly Needle, SAI Infusion Technology, Lake Villa, Ill., USA) was inserted in the cisterna magna to inject up to 80 μl of tracers (Gd-chelate: MultiHance, gadobenate dimeglumine; Bracco Diagnostics Inc, NJ, USA; 0.21 ml/kg) slowly over 30 seconds. To model the delivery of similar-sized therapeutic agents, two different sized molecules, free dye (IRDye800CW Carboxylate, LICOR, Lincoln, Nebr., USA; ˜1 kDa; 36 nmol/kg) and IR dye-conjugated antibody (Panitumumab-IRDye800: ˜150 kDa; 0.133 mg/kg) were co-delivered with the Gd-chelate. The respiratory rate (45-50 breaths per minute), and normal body temperature (36.5-37.5° C.) were maintained throughout the experiment through titration of isoflurane dose and with environmental heating.

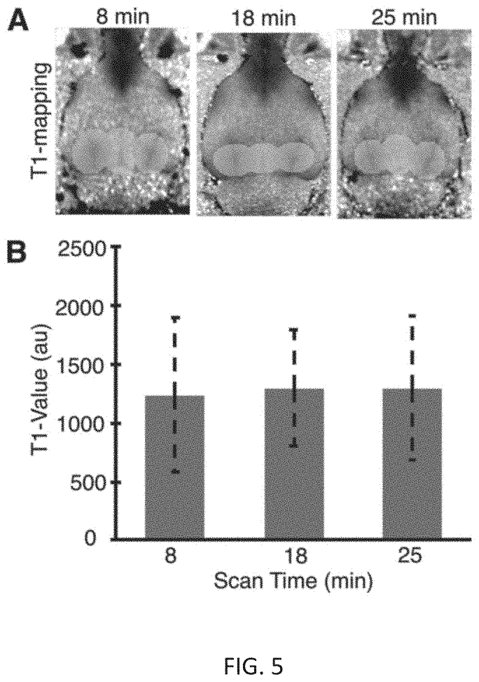

Magnetic Resonance Imaging Protocol. Magnetic Resonance Imaging (MRI) (an actively-shielded Bruker 7T horizontal bore scanner (Bruker Corp, Billerica Mass.), with International Electric Co. (IECO) gradient drivers, a 120 mm ID shielded gradient insert (600 mT/m, 1000 T/m/s), AVANCE III electronics; 8-channel multi-coil RF and multinuclear capabilities and volume RF coils; and the supporting Paravision 6.0.1 platform) was used to visualize glymphatic transport of Gd-chelate into the brain and to make quantitative T1-maps. The facility provides isoflurane anesthesia in medical-grade oxygen, and physiological monitoring of the subject including ECG, pulse oximetry, respiration, and temperature feedback for core body temperature maintenance by warm airflow over the animal. Following cisterna magna contrast agent injection, an MRI compatible animal FUS system (Image Guided Therapy—IGT, Pessac, France) was used in all experiments. Animals were placed in a prone position in a plastic stereotactic frame that has a single channel radiofrequency transmit-receive head coil (IGT) which is coupled to the FUS system. Animals were immobilized in the frame with ear bars and a bite bar. Noninvasive, MRI-compatible monitors for the respiratory rate and body temperature were used during the imaging session. Following localizer anatomical scout scans, a 3D T1-weighted (T1w) fast low angle shot (FLASH) sequence was acquired in the coronal plane with a repetition time (TR)=50 ms, echo time (TE)=3.53 ms, flip angle (FA)=20°, number of acquisition (NA)=1, field of view (FOV)=60×40×15 mm, slice thickness (ST)=0.3 mm, total scanning time=6 min 37 s, acquisition matrix size of 256×128×128 interpolated to 256×256×256, yielding an image resolution of 0.313×00.267×0.313 mm. A standard T1-map rapid acquisition with relaxation enhancement (RARE) with TE=7 ms, TR=300, 600, 1000, 1500, 2000, or 3000 ms, FOA=35×31 mm, ST=1 mm, scan time=8 min 58 s, image resolution=0.273×0.242 mm was acquired in a coronal plane to quantify Gd-diffusion within the brain parenchyma. Before setting up that standard T1-map sequence in the study, two more T1-map sequences were taken by replacing the last inversion time, 3000 ms by two different longer times, 4000 ms and 6000 ms to compare how T1-values changes between with cisternal injection of Gd (Table 1). Longer sequences did not make a significant change in T1-values at a constant volume so the standard sequence with last TR=3000 ms was used throughout the study (FIG. 5). T2* map-multiple gradient echo (MGE) weighted (T2*w) imaging with TR=796.76 ms at different time TE=3.50, 8.5, 13.5, 18.5, 23.5, 28.5, 33.5, 38.5, 43.5, 48.5 ms, FA=50°, FOV=60×40 mm, ST=1 mm, scan time=4 min 44 sec with image resolution=0.234×0.156 mm was used to image whether petechiae, which can result from excessive FUS exposures, occurred (FIG. 4). The scanning protocol consisted of the localizer, baseline, and post-FUS scans of 3D T1-FLASH, T1-map, and T2*w followed by intrathecal administration of Gd-chelate (MultiHance, gadobenate dimeglumine; Bracco Diagnostics Inc, NJ USA; 0.21 ml/kg) and/or co-delivery of Gd-chelate with optical tracers (Table 1). A total of 80 μl of the solution was delivered intrathecally using a 27-gauge butterfly catheter (SAI Infusion Technology) within a minute and the first baseline MRI acquisitions were imaged 12 min after the injection. FUS treatment was applied at 23 min after the intrathecal injection. All of the MRI acquisitions continued over either 2, 4, or 72 hours.

T1-mapping. In the first set of the experiment (N=26), 3D-T1w and T1-mapping MR images were taken to visualize CSF-ISF exchange of Gd-chelate into the brain and to quantify Gd-chelate kinetic parameters. The experimental timeline is shown in FIG. 2A and FIG. 5, 6. For quantitative measurements T1-map RARE protocol was set based on a RARE-sequence with one echo image, RARE factor=two and six T1 experiments. Each experiment has a different TR producing one image. By default, a T1-map is generated automatically for a single slice. Typical values for T1 of the rat brain can be found in previous publications. To achieve enough signal to noise ratio within a particular part of the organ, it is recommended to acquire several images so that they cover a time up to five times the T1 (79-83). To ensure agreement of T1-values with the literature, we first optimized the T1-mapping sequences within the hippocampal region of the brain at different spin-lattice relaxation time as shown in Table 1. Since T1-values at constant volume of the rat brain were not affected by different spin-lattice relaxation time, we decided to use a TR=3000 ms, 8 min scan time for further studies. A post-processing macro Fitinlsa which was implemented on the 7T Bruker Scanner to automatically start the T1 parameters map calculation to extract quantitative measurements of T1-mapping.

### Analysis

T1 was calculated from the Image Sequence Analysis (ISA) functiont1sat:

$$Y = A + C x [ 1 - exp ( - \frac{t}{T 1} ) ]$$

The parameters are defined in the following way:
A—absolute bias,
C—signal intensity,
T1—spin-lattice relaxation time.

This function supplied by Bruker uses a repetition time list calculated from the protocol parameters to generate the T1 relaxation curve. The fit is based on the magnitude image of the reconstructed dataset. OsiriX 10.0.5 was used to calculate the Gd-enhanced brain volume over time after cisternal Gd-chelate injection using the T1-mapping sequences.

Focused Ultrasound System. For good acoustic coupling of the FUS beam and for getting access to the cisterna magna for intrathecal injection, the dorsal scalp fur in the sonication trajectory plus up to 3 cm towards neck was removed using standard hair removal cream. After the cisternal injection, animals were placed in a plastic stereotactic frame that is coupled to an MR compatible FUS system (Image Guided Therapy—IGT, Pessac, France), and immobilized with ear bars and a bite bar. A thin layer of ultrasound gel was applied to pair the water-filled coupling membrane of the FUS transducer to the skin of the head. Once the anesthetized animal was secured in the holder and the transducer was placed over its head, approximately at the center of the brain, the assembly was inserted in the bore of the MRI scanner. The sonication trajectory was selected using the remote positioning capabilities of the transducer in all three axes. Stereotactic coordinates for sonication are shown in FIG. 1B, right. Briefly, an 8×10 mm black-dotted rectangle centered on the brain is selected with corners starting from 4 mm lateral at the bregma region (4,0) and move 8 mm to left at (−4,0) then 10 mm posterior at (−4, −10) and then move 8 mm to the right at (4, −10) as indicated in FIG. 1B. Ultrasound was held on continuously while the transducer slowly moved around the FUS trajectory for 10 min. Total time to complete one trajectory loop: 24 sec, with a 50 ms pause time between each loop, with 25 total loops for each rat. For sham procedures, the same positioning and trajectory was chosen but the power to the ultrasound transducer was disconnected. FUS (0.25 MI in situ, ˜ 7.7% duty cycle for 10 min) or sham was applied transcranially throughout the brain. The orange rectangle represents the expected ultrasound exposure zone based on the ultrasound transducer's focal spot size (2.78×12 mm) that covers a significant part of the rat brain. To account for skull attenuation, a 30% pressure insertion loss was assumed for this size and age of rats. The expected volume coverage region in rat brain by a single sonication using this particular transducer can be envisioned based on our previous paper (FIG. 5).

Fluorescence Imaging. In the second set of experiments (N=13), two different sized molecules, free dye (IRDye8000W: 1 kDa) and IR dye-conjugated antibody (Panitumumab-IRDye800: 150 kDa) were co-delivered with Gd-chelate to model the delivery of similar-sized therapeutic agents. The experimental timeline can be found in FIG. 3A. Briefly, dyes were diluted with Gd-chelate and injected intrathecally at 0 min. A 3D T1w image was used to confirm the cisternal injection and FUS (0.25 MI in situ, 2% duty cycle for 10 min) or sham was applied transracially throughout the brain. About 2 hours after the intrathecal delivery, animals were euthanized with an overdose of euthasol. Then the rat brain was flash frozen in dry ice with 2-methylbutane (Fisher, Pittsburgh, Pa.). For tissue sectioning, the frozen rat brain was mounted with a minimal amount of optimal cutting medium (OCT) compound and sectioned at a 20 μm thickness using a cryostat (LEICA CM 1950, Buffalo Grove, Ill., USA)). Every 10^{th} section (200 μm apart) was saved for optical imaging. The specimen temperature was set at −19° C. and the chamber temperature at −20° C. Tissue sections were thaw-mounted on microscope glass slides (Fisher, Pittsburgh, Pa.), fixed the tissue-slides using 4% paraformaldehyde, and applied DAPI for fluorescence imaging. All fluorescence images were collected in a near-infrared fluorescence imager (Ex/Em: 785/820 nm, Pearl Trilogy Imaging System, LI-COR) with 85 μm resolution and processed with Image Studio (version 5.2, LI-COR).

Hematoxylin and Eosin Staining Histology. In the third set of the experiment (N=3), T2*w images, as well as hematoxylin and eosin (H & E) staining, were used to evaluate for parenchymal damage. The experimental timeline is shown in FIG. 4A. Two hours after the Gd-chelate injection, animals were sacrificed and the brain fixed via transcardial perfusion (0.9% NaCl, 100 mL; 10% buffered formalin phosphate, 250 mL). The brain was then removed, embedded in paraffin, and serially sectioned at 5 μm in the axial plane (perpendicular to the direction of ultrasound beam propagation). Every 50^{th} section (250 μm apart) was stained with H&E.

Quantification and Statistical Analysis. All MR-images were analyzed in OsiriX (version 10.0.5). An axial plane of T1-mapp sequence with 5/1700 as a lower/upper threshold was used for manual ROI segmentation to calculate the volume of brain parenchymal penetration of the gadolinium tracer. All IRDye images were analyzed in Image Studio (version 5.2, LI-COR). Four-five slices were included from each animal to the analysis. Signals above the background was used for manual ROI segmentation to calculate the area of brain parenchyma penetration of the dye. All the data that were generated from the imaging software were plotted using Microsoft Excel (version 16.16.22). All values were presented as mean±standard deviation. Statistical analyses were performed with Microsoft Excel (version 16.16.22) and JMP (version 13.2.1). Two-tailed paired Student's t-test was used to compare the gadolinium-enhanced volume and IRDye-enhanced area between sonicated and non-sonicated (Sham) groups. One-way ANOVA with post-hoc Tukey-Kramer tests were used to compare gadolinium-enhanced volume at different time points (12 min, 35 min, 70 min, 105 min, 180 min, 240 min) within the same group, either Ultrasound or Sham. P-values<0.05 were considered statically significant.

T1-mapping. As mentioned in the methods, we first optimized the T1-mapping sequences using the values measured of the hippocampal region of the brain with different spin-lattice relaxation times. We found T1-values across a constant volume (0.33 cm³) within the hippocampal region of the rat brain to be 1235±657, 1299±502, and 1298±618 with the longest TR=3000, 4000, and 6000 ms respectively (Table 1). These T1-values are similar to T1-values that are published already in the literature for this particular magnetic strength, 7T (1-3), and are relatively similar to each other (FIG. 5). To minimize MRI scan time, we decided to use the protocol with 8 min total scan time and TR=3000 ms for the rest of these studies. As observed in the plot in FIG. 2D, the mean gadolinium-enhanced volume in the sham cohort was 0.17±0.084, 0.25±0.15, 0.22±0.1, 0.23±0.11, 0.11±0.12 and 0.06±0.04 and in the Ultrasound cohort was 0.18±0.07, 0.43±0.16, 0.44±0.17, 0.45±0.25, 0.18±0.24 and 0.17±0.24 at 12 min, 35 min, 70 min, 105 min, 180 min and 240 min respectively.

Ultrasonic glymphatic induction is safe. The penetration of the MRI tracer into the brain and the presence or lack of petechiae were confirmed using contrast-enhanced T1w and T2*w MRI, respectively (FIGS. 4B and S2). FIG. S2A shows the parenchymal uptake of Gd-chelate represented by a pseudo-color T1w MR image. The long-term effects of the ultrasound intervention would show on T2*w MR images for up to 72 hours. We observed in the T1w images in FIG. 2B that the Gd-chelate cleared from the CSF-ISF spaces by 3 hours. Here, we further confirmed that there was no any evidence of Gd deposition (FIG. S-2A) in 24-72 hours. T2*w revealed that there was no long-term adverse effect such as edema or hemorrhage after the brain-wide ultrasound exposure with 0.25 MI in situ, ˜7.7% duty cycle for 10 min (FIG. S-2B). By using the Gd-chelate clearance pattern from the prior experiments, we designed a second set of experimental safety evaluations where we monitored animals in the MRI up to 2 hours and proceed to ex vivo histological evaluation.

### REFERENCES

1. E. Neuwelt, N. J. Abbott, L. Abrey, W. A. Banks, B. Blakley, T. Davis, B. Engelhardt, P. Grammas, M. Nedergaard, J. Nutt, W. Pardridge, G. A. Rosenberg, Q. Smith, L. R. Drewes, Strategies to advance translational research into brain barriers, The Lancet Neurology 7, 84-96 (2008).
2. N. J. Abbott, I. A. Romero, Transporting therapeutics across the blood-brain barrier, Mol Med Today 2, 106-113 (1996).
3. W. M. Pardridge, Molecular biology of the blood-brain barrier, Mol. Biotechnol. 30, 57-70 (2005).
4. S. Jain, M. Malinowski, P. Chopra, V. Varshney, T. R. Deer, Intrathecal drug delivery for pain management: recent advances and future developments, Expert Opin Drug Deliv 16, 815-822 (2019).
5. P. Calias, W. A. Banks, D. Begley, M. Scarpa, P. Dickson, Intrathecal delivery of protein therapeutics to the brain: a critical reassessment, Pharmacol. Ther. 144, 114-122 (2014).
6. T. Leal, J. E. Chang, M. Mehta, H. I. Robins, Leptomeningeal Metastasis: Challenges in Diagnosis and Treatment, Curr Cancer Ther Rev 7, 319-327 (2011).
7. P. A. Burch, S. A. Grossman, C. S. Reinhard, Spinal cord penetration of intrathecally administered cytarabine and methotrexate: a quantitative autoradiographic study, J. Natl. Cancer Inst. 80, 1211-1216 (1988).
8. J. J. Iliff, H. Lee, M. Yu, T. Feng, J. Logan, M. Nedergaard, H. Benveniste, Brain-wide pathway for waste clearance captured by contrast-enhanced MRI, J Clin Invest 123, 1299-1309 (2013).
9. J. J. Iliff, M. Wang, Y. Liao, B. A. Plogg, W. Peng, G. A. Gundersen, H. Benveniste, G. E. Vates, R. Deane, S. A. Goldman, E. A. Nagelhus, M. Nedergaard, A paravascular pathway facilitates CSF flow through the brain parenchyma and the clearance of interstitial solutes, including amyloid β, Sci Transl Med 4, 147ra111 (2012).
10. M. K. Rasmussen, H. Mestre, M. Nedergaard, The glymphatic pathway in neurological disorders, Lancet Neurol 17, 1016-1024 (2018).
11. J. H. Ahn, H. Cho, J.-H. Kim, S. H. Kim, J.-S. Ham, I. Park, S. H. Suh, S. P. Hong, J.-H. Song, Y.-K. Hong, Y. Jeong, S.-H. Park, G. Y. Koh, Meningeal lymphatic vessels at the skull base drain cerebrospinal fluid, Nature 572, 62-66 (2019).
12. A. Louveau, I. Smirnov, T. J. Keyes, J. D. Eccles, S. J. Rouhani, J. D. Peske, N. C. Derecki, D. Castle, J. W. Mandell, K. S. Lee, T. H. Harris, J. Kipnis, Structural and functional features of central nervous system lymphatic vessels, Nature 523, 337-341 (2015).
13. S. D. Mesquita, Z. Fu, J. Kipnis, The Meningeal Lymphatic System: A New Player in Neurophysiology, Neuron 100, 375-388 (2018).
14. H. Benveniste, X. Liu, S. Koundal, S. Sanggaard, H. Lee, J. Wardlaw, The Glymphatic System and Waste Clearance with Brain Aging: A Review, GER 65, 106-119 (2019).
15. L. Yang, B. T. Kress, H. J. Weber, M. Thiyagarajan, B. Wang, R. Deane, H. Benveniste, J. J. Iliff, M. Nedergaard, Evaluating glymphatic pathway function utilizing clinically relevant intrathecal infusion of CSF tracer, J Transl Med 11, 107 (2013).
16. N. A. Jessen, A. S. F. Munk, I. Lundgaard, M. Nedergaard, The Glymphatic System: A Beginner's Guide, Neurochem Res 40, 2583-2599 (2015).
17. C. A. Hawkes, N. Jayakody, D. A. Johnston, I. Bechmann, R. O. Carare, Failure of perivascular drainage of β-amyloid in cerebral amyloid angiopathy, Brain Pathol. 24, 396-403 (2014).
18. B. T. Kress, J. J. Iliff, M. Xia, M. Wang, H. Wei, D. Zeppenfeld, L. Xie, H. Kang, Q. Xu, J. Liew, B. A. Plog, F. Ding, R. Deane, M. Nedergaard, Impairment of paravascular clearance pathways in the aging brain, Ann Neurol 76, 845-861 (2014).
19. W. Peng, T. M. Achariyar, B. Li, Y. Liao, H. Mestre, E. Hitomi, S. Regan, T. Kasper, S. Peng, F. Ding, H. Benveniste, M. Nedergaard, R. Deane, Suppression of glymphatic fluid transport in a mouse model of Alzheimer's disease, Neurobiol. Dis. 93, 215-225 (2016).
20. J. J. Iliff, M. J. Chen, B. A. Plog, D. M. Zeppenfeld, M. Soltero, L. Yang, I. Singh, R. Deane, M. Nedergaard, Impairment of Glymphatic Pathway Function Promotes Tau Pathology after Traumatic Brain Injury, J Neurosci 34, 16180-16193 (2014).
21. R. Goulay, J. Flament, M. Gauberti, M. Naveau, N. Pasquet, C. Gakuba, E. Emery, P. Hantraye, D. Vivien, R. Aron-Badin, T. Gaberel, Subarachnoid Hemorrhage Severely Impairs Brain Parenchymal Cerebrospinal Fluid Circulation in Nonhuman Primate, Stroke 48, 2301-2305 (2017).
22. T. Gaberel, C. Gakuba, R. Goulay, S. Martinez De Lizarrondo, J.-L. Hanouz, E. Emery, E. Touze, D. Vivien, M. Gauberti, Impaired glymphatic perfusion after strokes revealed by contrast-enhanced MRI: a new target for fibrinolysis?, Stroke 45, 3092-3096 (2014).
23. H. Chen, E. E. Konofagou, The size of blood-brain barrier opening induced by focused ultrasound is dictated by the acoustic pressure, J. Cereb. Blood Flow Metab. 34, 1197-1204 (2014).
24. H. Chen, C. C. Chen, C. Acosta, S.-Y. Wu, T. Sun, E. E. Konofagou, A New Brain Drug Delivery Strategy: Focused Ultrasound-Enhanced Intranasal Drug Delivery, 9, el 08880 (2014).
25. K.-C. Wei, P.-C. Chu, H.-Y. J. Wang, C.-Y. Huang, P.-Y. Chen, H.-C. Tsai, Y.-J. Lu, P.-Y. Lee, I.-C. Tseng, L.-Y. Feng, P.-W. Hsu, T.-C. Yen, H.-L. Liu, Focused Ultrasound-Induced Blood-Brain Barrier Opening to Enhance Temozolomide Delivery for Glioblastoma Treatment: A Preclinical Study, PLoS ONE 8, e58995 (2013).
26. M. Aryal, J. Park, N. Vykhodtseva, Y.-Z. Zhang, N. McDannold, Enhancement in blood-tumor barrier permeability and delivery of liposomal doxorubicin using focused ultrasound and microbubbles: evaluation during tumor progression in a rat glioma model, Phys Med Biol60, 2511-2527 (2015).
27. J. B, H. H, L. Eh, H. M, P. J, An advanced focused ultrasound protocol improves the blood-brain barrier permeability and doxorubicin delivery into the rat brain, J Control Release 315, 55-64(2019).
28. A. B. Etame, R. J. Diaz, M. A. O'Reilly, C. A. Smith, T. G. Mainprize, K. Hynynen, J. T. Rutka, Enhanced delivery of gold nanoparticles with therapeutic potential into the brain using MRI-guided focused ultrasound, Nanomedicine 8, 1133-1142 (2012).
29. A. Mohammadabadi, R. N. Huynh, A. S. Wadajkar, R. G. Lapidus, A. J. Kim, C. B. Raub, V. Frenkel, Pulsed focused ultrasound lowers interstitial fluid pressure and increases nanoparticle delivery and penetration in head and neck squamous cell carcinoma xenograft tumors, Phys Med Biol65, 125017 (2020).
30. B. P. Mead, C. T. Curley, N. Kim, K. Negron, W. J. Garrison, J. Song, D. Rao, G. W. Miller, J. W. Mandell, B. W. Purow, J. S. Suk, J. Hanes, R. J. Price, Focused Ultrasound Preconditioning for Augmented Nanoparticle Penetration and Efficacy in the Central Nervous System, Small 15, 1903460 (2019).
31. C. T. Curley, B. P. Mead, K. Negron, N. Kim, W. J. Garrison, G. W. Miller, K. M. Kingsmore, E. A. Thim, J. Song, J. M. Munson, A. L. Klibanov, J. S. Suk, J. Hanes, R. J. Price, Augmentation of brain tumor interstitial flow via focused ultrasound promotes brain-penetrating nanoparticle dispersion and transfection, Science Advances 6, eaay1344 (2020).
32. C. Gakuba, T. Gaberel, S. Goursaud, J. Bourges, C. Di Palma, A. Quenault, S. M. de Lizarrondo, D. Vivien, M. Gauberti, General Anesthesia Inhibits the Activity of the “Glymphatic System,” Theranostics 8, 710-722 (2018).
33. A. R. Mendelsohn, J. W. Larrick, Sleep Facilitates Clearance of Metabolites from the Brain: Glymphatic Function in Aging and Neurodegenerative Diseases, Rejuvenation Research 16, 518-523 (2013).
34. T. Mainprize, N. Lipsman, Y. Huang, Y. Meng, A. Bethune, S. Ironside, C. Heyn, R. Alkins, M. Trudeau, A. Sahgal, J. Perry, K. Hynynen, Blood-Brain Barrier Opening in Primary Brain Tumors with Non-invasive MR-Guided Focused Ultrasound: A Clinical Safety and Feasibility Study, Sci Rep 9, 321 (2019).
35. Basics of Biomedical Ultrasound for Engineers|Wiley Wiley.com (available at https://www.wiley.com/en-us/Basics+of+Biomedical+Ultrasound+for+Engineers-p-9780470465479).
36. W. L. Nyborg, Solutions of the bio-heat transfer equation, Phys. Med. Biol. 33, 785-792 (1988).
37. D. >Gail ter Haar, C. Coussios, High intensity focused ultrasound: Physical principles and devices, International Journal of Hyperthermia 23, 89-104 (2007).
38. The Rat Brain in Stereotaxic Coordinates—7th Edition (available at https://www.elsevier.com/books/the-rat-brain-in-stereotaxic-coordinates/paxinos/978-O-12-391949-6).
39. R. W. Gao, N. Teraphongphom, E. de Boer, N. S. van den Berg, V. Divi, M. J. Kaplan, N. J. Oberhelman, S. S. Hong, E. Capes, A. D. Colevas, J. M. Warram, E. L. Rosenthal, Safety of panitumumab-IRDye800CW and cetuximab-IRDye800CW for fluorescence-guided surgical navigation in head and neck cancers, Theranostics 8, 2488-2495 (2018).
40. C. H. Liu, H. E. D'Arceuil, A. J. de Crespigny, Direct CSF injection of MnCl2 for dynamic manganese-enhanced MRI, Magnetic Resonance in Medicine 51, 978-987 (2004).
41. H. Lee, K. Mortensen, S. Sanggaard, P. Koch, H. Brunner, B. Quistorff, M. Nedergaard, H. Benveniste, Quantitative Gd-DOTA uptake from cerebrospinal fluid into rat brain using 3D VFA-SPGR at 9.4 T, Magn Reson Med 79, 1568-1578 (2018).
42. T. R. Nelson, J. B. Fowlkes, J. S. Abramowicz, C. C. Church, Ultrasound Biosafety Considerations for the Practicing Sonographer and Sonologist, Journal of Ultrasound in Medicine 28, 139-150 (2009).
43. Y. Chen, H. Imai, A. Ito, N. Saito, Novel modified method for injection into the cerebrospinal fluid via the cerebellomedullary cistern in mice, 8.
44. C. S. Edeklev, M. Halvorsen, G. Løvland, S. a. S. Vatnehol, Ø. Gjertsen, B. Nedregaard, R. Sletteberg, G. Ringstad, P. K. Eide, Intrathecal Use of Gadobutrol for Glymphatic MR Imaging: Prospective Safety Study of 100 Patients, American Journal of Neuroradiology 40, 1257-1264 (2019).
45. P. K. Eide, S. A. S. Vatnehol, K. E. Emblem, G. Ringstad, Magnetic resonance imaging provides evidence of glymphatic drainage from human brain to cervical lymph nodes, Sci Rep 8, 1-10 (2018).
46. F. Mack, B. G. Baumert, N. Schafer, E. Hattingen, B. Scheffler, U. Herrlinger, M. Glas, Therapy of leptomeningeal metastasis in solid tumors, Cancer Treat. Rev. 43, 83-91 (2016).
47. N. J. Abbott, M. E. Pizzo, J. E. Preston, D. Janigro, R. G. Thorne, The role of brain barriers in fluid movement in the CNS: is there a “glymphatic” system?, Acta Neuropathol. 135, 387-407 (2018).
48. A. Aspelund, S. Antila, S. T. Proulx, T. V. Karlsen, S. Karaman, M. Detmar, H. Wiig, K. Alitalo, A dural lymphatic vascular system that drains brain interstitial fluid and macromolecules, J. Exp. Med. 212, 991-999 (2015).
49. I. Lundgaard, M. L. Lu, E. Yang, W. Peng, H. Mestre, E. Hitomi, R. Deane, M. Nedergaard, Glymphatic clearance controls state-dependent changes in brain lactate concentration, J Cereb Blood Flow Metab 37, 2112-2124 (2017).
50. S. Mader, L. Brimberg, Aquaporin-4 Water Channel in the Brain and Its Implication for Health and Disease, Cells 8, 90 (2019).
51. B. A. Plog, M. Nedergaard, The Glymphatic System in Central Nervous System Health and Disease: Past, Present, and Future, Annual Review of Pathology: Mechanisms of Disease 13, 379-394 (2018).
52. T. Pu, W. Zou, W. Feng, Y. Zhang, L. Wang, H. Wang, M. Xiao, Persistent Malfunction of Glymphatic and Meningeal Lymphatic Drainage in a Mouse Model of Subarachnoid Hemorrhage, Experimental Neurobiology 28, 104 (2019).
53. M. J. Simon, J. J. Iliff, Regulation of cerebrospinal fluid (CSF) flow in neurodegenerative, neurovascular and neuroinflammatory disease, Biochimica et Biophysica Acta (BBA)—Molecular Basis of Disease 1862, 442-451 (2016).
54. B. Bein, P. Meybohm, E. Cavus, P. H. Tonner, M. Steinfath, J. Scholz, V. Doerges, A comparison of transcranial Doppler with near infrared spectroscopy and indocyanine green during hemorrhagic shock: a prospective experimental study, Critical Care 10, R18 (2006).
55. Y. Chen, W. Xu, L. Wang, X. Yin, J. Cao, F. Deng, Y. Xing, J. Feng, Transcranial Doppler combined with quantitative EEG brain function monitoring and outcome prediction in patients with severe acute intracerebral hemorrhage, Critical Care 22, 36 (2018).
56. G. Meijler, S. J. Steggerda, in Neonatal Cranial Ultrasonography, G. Meijler, S. J. Steggerda, Eds. (Springer International Publishing, Cham, 2019), pp. 219-257.
57. J. Naqvi, K. H. Yap, G. Ahmad, J. Ghosh, Transcranial Doppler Ultrasound: A Review of the Physical Principles and Major Applications in Critical Care International Journal of Vascular Medicine 2013, e629378 (2013).
58. C. L. Onweni, D. C. McLaughlin, W. D. Freeman, How I use transcranial Doppler in the ICU, Critical Care 24, 38 (2020).
59. S. Purkayastha, F. Sorond, Transcranial Doppler Ultrasound: Technique and Application, Semin Neurol 32, 411-420 (2012).
60. F. A. Rasulo, R. Bertuetti, C. Robba, F. Lusenti, A. Cantoni, M. Bernini, A. Girardini, S. Calza, S. Piva, N. Fagoni, N. Latronico, The accuracy of transcranial Doppler in excluding intracranial hypertension following acute brain injury: a multicenter prospective pilot study, Critical Care 21, 44 (2017).
61. S. Sarkar, S. Ghosh, S. K. Ghosh, A. Collier, Role of transcranial Doppler ultrasonography in stroke, Postgrad Med J 83, 683-689 (2007).
62. A. Bystritsky, A. S. Korb, P. K. Douglas, M. S. Cohen, W. P. Melega, A. P. Mulgaonkar, A. DeSalles, B.-K. Min, S.-S. Yoo, A review of low-intensity focused ultrasound pulsation, Brain Stimulation 4, 125-136 (2011).
63. M. Fini, W. J. Tyler, Transcranial focused ultrasound: a new tool for non-invasive neuromodulation, International Review of Psychiatry 0, 1-10 (2017).
64. P. Ghanouni, K. B. Pauly, W. J. Elias, J. Henderson, J. Sheehan, S. Monteith, M. Wintermark, Transcranial MRI-Guided Focused Ultrasound: A Review of the Technologic and Neurologic Applications, American Journal of Roentgenology 205, 150-159 (2015).
65. D. S. Hersh, A. J. Kim, J. A. Winkles, H. M. Eisenberg, G. F. Woodworth, V. Frenkel, Emerging Applications of Therapeutic Ultrasound in Neuro-oncology: Moving Beyond Tumor Ablation, Neurosurgery (2016), doi:10.1227/NEU.0000000000001399.
66. K. Hynynen, N. McDannold, MRI guided and monitored focused ultrasound thermal ablation methods: a review of progress, Int J Hyperthermia 20, 725-737 (2004).
67. Z. Izadifar, Z. Izadifar, D. Chapman, P. Babyn, An Introduction to High Intensity Focused Ultrasound: Systematic Review on Principles, Devices, and Clinical Applications, J Clin Med 9 (2020), doi:10.3390/jcm9020460.
68. F. A. Jolesz, MRI-Guided Focused Ultrasound Surgery, Annual Review of Medicine 60, 417-430 (2009).
69. L. Lamsam, E. Johnson, I. D. Connolly, M. Wintermark, M. Hayden Gephart, A review of potential applications of MR-guided focused ultrasound for targeting brain tumor therapy, Neurosurgical Focus 44, E10 (2018).
70. R. Medel, S. J. Monteith, W. J. Elias, M. Eames, J. Snell, J. P. Sheehan, M. Wintermark, F. A. Jolesz, N. F. Kassell, Magnetic Resonance Guided Focused Ultrasound Surgery: Part 2—A Review of Current and Future Applications, Neurosurgery 71, 755-763 (2012).
71. C. Poon, D. McMahon, K. Hynynen, Noninvasive and targeted delivery of therapeutics to the brain using focused ultrasound, Neuropharmacology 120, 20-37 (2017).
72. N. Vykhodtseva, N. McDannold, K. Hynynen, PROGRESS AND PROBLEMS IN THE APPLICATION OF FOCUSED ULTRASOUND FOR BLOOD-BRAIN BARRIER DISRUPTION, Ultrasonics 48, 279-296 (2008).
73. A. Abrahao, Y. Meng, M. Llinas, Y. Huang, C. Hamani, T. Mainprize, I. Aubert, C. Heyn, S. E. Black, K. Hynynen, N. Lipsman, L. Zinman, First-in-human trial of blood-brain barrier opening in amyotrophic lateral sclerosis using MR-guided focused ultrasound, Nature Communications 10, 4373 (2019).
74. C. D. Arvanitis, M. S. Livingstone, N. McDannold, Combined Ultrasound and MR Imaging to Guide Focused Ultrasound Therapies in the Brain, Phys Med Biol 58, 4749-4761 (2013).
75. A. N. Pouliopoulos, S.-Y. Wu, M. T. Burgess, M. E. Karakatsani, H. A. S. Kamimura, E. E. Konofagou, A Clinical System for Non-invasive Blood-Brain Barrier Opening Using a Neuronavigation-Guided Single-Element Focused Ultrasound Transducer, Ultrasound in Medicine & Biology 46, 73-89 (2020).
76. L. Liu, K. Duff, A Technique for Serial Collection of Cerebrospinal Fluid from the Cisterna Magna in Mouse, Journal of Visualized Experiments (2008), doi:10.3791/960.
77. T. G. dos Santos, M. S. L. Pereira, D. L. Oliveira, Rat Cerebrospinal Fluid Treatment Method through Cisterna Cerebellomedullaris Injection, Neurosci. Bull., 1-6 (2018).
78. M. Behroozi, C. Chwiesko, F. Ströckens, M. Sauvage, X. Helluy, J. Peterburs, O. Güntürkün, In vivo measurement of T1 and T2 relaxation times in awake pigeon and rat brains at 7 T, Magn Reson Med 79, 1090-1100 (2018).
79. D. N. Guilfoyle, V. V. Dyakin, J. O'Shea, G. S. Pell, J. A. Helpern, Quantitative measurements of proton spin-lattice (T1) and spin-spin (T2) relaxation times in the mouse brain at 7.0 T, Magnetic Resonance in Medicine 49, 576-580 (2003).
80. R. A. de Graaf, P. B. Brown, S. McIntyre, T. W. Nixon, K. L. Behar, D. L. Rothman, High magnetic field water and metabolite proton T1 and T2 relaxation in rat brain in vivo, Magn Reson Med 56, 386-394 (2006).
81. A. M. Chow, D. S. Gao, S. J. Fan, Z. Qiao, F. Y. Lee, J. Yang, K. Man, E. X. Wu, Measurement of liver T₁ and T₂ relaxation times in an experimental mouse model of liver fibrosis, J Magn Reson Imaging 36, 152-158 (2012).
82. Focal brain ischemia in rat: acute changes in brain tissue T1 reflect acute increase in brain tissue water content.-Abstract-Europe PMC (available at https://europepmc.org/article/med/16206135).
83. W. Lin, R. Venkatesan, K. Gurleyik, Y. Y. He, W. J. Powers, C. Y. Hsu, An absolute measurement of brain water content using magnetic resonance imaging in two focal cerebral ischemic rat models, J. Cereb. Blood Flow Metab. 20, 37-44 (2000).
84. M. A. O'Reilly, A. Muller, K. Hynynen, Ultrasound insertion loss of rat parietal bone appears to be proportional to animal mass at submegahertz frequencies, Ultrasound Med Biol 37, 1930-1937 (2011).
85. J. B. Wang, M. Aryal, Q. Zhong, D. B. Vyas, R. D. Airan, Noninvasive Ultrasonic Drug Uncaging Maps Whole-Brain Functional Networks, Neuron 100, 728-738.e7 (2018).

What is claimed is:

1. A method of increasing brain penetration of an intrathecally administered agent, the method comprising:
intrathecally administering an agent to a subject; and
applying transcranial ultrasound to the subject to modulate the glymphatic pathway to promote brain penetration of the intrathecally administered agent.

2. The method of claim 1, wherein the method comprises applying transcranial ultrasound across the whole brain of the subject.

3. The method of claim 1, wherein the transcranial ultrasound has a frequency ranging from 600 kHz to 700 kHz.

4. The method of claim 1, wherein the transcranial ultrasound has an intensity ranging from 10 mW/cm² to 450 mW/cm².

5. The method of claim 1, wherein the transcranial ultrasound has a mechanical index ranging from 0.2 to 0.3.

6. The method of claim 1, wherein the transcranial ultrasound comprises transcranial focused ultrasound.

7. The method of claim 1, wherein the method comprises upregulating the glymphatic pathway.

8. The method of claim 1, wherein the agent has a molecular weight ranging from 1 kDa to 150 kDa.

9. The method of claim 1, wherein the agent is a small molecule.

10. The method of claim 1, wherein the agent is an antibody or fragment thereof.

11. The method of claim 1, wherein the agent is a nucleic acid.

12. The method of claim 1, wherein brain penetration is increased by 60% to 110% compared to a control.
